(19) United States
(12) Patent Application Publication
(10) Pub. No.: US 2004/0072815 A1
(43) Pub. Date: Apr. 15, 2004

(54) Antibiotic composition and method

(76) Inventor: Gary A. Koppel, Indianapolis, IN (US)

Correspondence Address: Barnes & Thornburg, 11 South Meridian Street, Indianapolis, IN, 46204, US

(21) Appl. No.: 10/467,185

(22) PCT Filed: Feb. 12, 2002
(86) PCT No.: PCT/US02/04230

(51) Int. Cl. A61K 31/43; A61K 31/545

(52) U.S. Cl. 514/192; 514/200

(57) ABSTRACT

Novel antibiotic compositions are described. β-lactam antibiotics are used in combination with a non-β-lactam inhibitor of NAALADase, the inhibitor in an amount effective to inhibit β-lactamase activity. The antibiotic compositions can be administered to treat infections caused by β-lactamase-producing bacterial strains.

### FIELD OF INVENTION

This application relates to antibiotic compositions and their use for treating bacterial infections. More particularly, this invention is directed to antibiotic compositions comprising β-lactam antibiotics and a non-β-lactam peptidase inhibitor and use of such compositions for treating bacterial infections.

### BACKGROUND OF THE INVENTION

Clavulanic acid is one of several β-lactamase inhibitors used commercially in combination with certain β-lactamase susceptible β-lactam antibacterials. It is a potent inhibitor of “serine” β-lactamase and is used commercially as its potassium salt in conjunction with amoxicillin and with ticarcillin. Structurally, clavulanic acid, as well as the other β-lactamase inhibitors finding commercial use are bicyclic compounds comprising a β-lactam ring system. Clavulanic acid itself has only weak, though broad spectrum, antibacterial activity. Its antibacterial mode of action is via inhibition of penicillin binding protein 2. It is absorbed and distributed in any number of animal species, including man. There have been extensive studies on the kinetics and mechanism of interaction of various β-lactamases with clavulanic acid and of inactivation of clavulanic acid. Most experiments have used TEM β-lactamase from E. coli, although β-lactamases from other β-lactamase producing microorganisms have also been investigated and show a high degree of active site homology, including a conserved nucleophilic serine residue. Clavulanic acid has a very high binding affinity for the serine β-lactamases. E. coli TEM-β-lactamase has been cloned, sequenced and crystallized to determine the active site motifs. The four putative binding sites on TEM-β-lactamase that could accommodate clavulanic acid are designated sites I, II, III, and IV. These sites are likely part of the active site and their location and amino acid (AA) sequences are as follows:

Site I:

35 AA's downstream from N-terminus: STTK

Site II:

57 AA's downstream from STTK motif: SGC, SGN, or SAN

Site III:

111 AA's downstream from SGC motif: KTG

Site IV:

41 AA's downstream from SGC motif: ENKD

Recently, Applicant has discovered that clavulanic acid is a potent inhibitor of the enzyme glutamylcarboxy peptidase, specifically N-acetylated, α-linked, acidic dipeptidase (NAALADase). Comparison of the putative active site motifs of serine β-lactamase and the amino acid sequences of human and rat NAALADase revealed a similar positioning and a high degree of homology between the putative active site motifs of β-lactamase and of both human and rat NAALADase. The similarities between β-lactamase and NAALADase is shown below:

Site I:

Beta-lactamase: 35 AA's downstream from N-terminus: STTK

NAALADase: 38 AA's downstream from N-terminus: STQK

Site II:

Beta-lactamase: 57 AA's downstream from STTK motif: SGC, SGN, or SAN

NAALADase: 59 AA's downstream from STQK motif: SFG

Site III:

Beta-lactamase: 111 AA's downstream from SGC motif: KTG

NAALADase: 110 AA's downstream from SFG motif: KLG

Site IV:

Beta-lactamase: 41 AA's downstream from SGC motif: ENKD

NAALADase: 41 AA's downstream from SFG motif: ERGV

Given the high affinity of clavulanic acid for NAALADase and serine β-lactamase and further in view of the significant amino acid sequence homology between the β-lactamase active site motif and key sequences of NAALADase, it can be inferred that the binding properties of other β-lactamase inhibitors to NAALADase would be similar or that such homology is a molecular signature of an enzyme derived from beta-lactamase, such as NAALADase, which may bind inhibitors by some other modified active site motif. Further, one can infer that compounds known to exhibit affinity for NAALADase and known to inhibit NAALADase activity, including many non-β-lactam compounds, also, like clavulanic acid, inhibit serine β-lactamases.

Thus in accordance with this invention there is provided an antibiotic composition comprising a β-lactam antibiotic, a non-β-lactam inhibitor of N-acetylated, α-linked, acetic peptidase in an amount effective to inhibit β-lactamase, and a pharmaceutically acceptable carrier. Similarly the present invention affords a method for treating bacterial infections, particularly those of β-lactamase-producing bacteria by administering a β-lactam antibiotic and a non-β-lactam containing inhibitor of a N-acetylated α-linked acetic peptidase in an amount effective to inhibit β-lactamase. The invention, again, is based on the probative active site motif amino acid sequence homology between NAALADase and bacterial β-lactamases.

### DETAILED DESCRIPTION OF THE INVENTION

β-lactam antibiotics have enjoyed widespread commercial use in the treatment of bacterial infections in animals, including man. The literature is replete with references to the preparation, use and clinical applications of such antibiotic compounds. Structurally such compounds have in common a β-lactam ring structure and are in general terms either monocyclic β-lactam compounds or bicyclic β-lactam compounds, including generally penams, cephems, 1-oxa-1-dethiacephems, clavams, clavems, carbapenams, carbapenems, and carbacephems. The most common β-lactam antibiotics in commercial use today are penicillins and cephalosporins. Examples of such compounds are:

cefamandole nafate (Mandol)

cefazolin sodium (Zolicef, Ancef, Kefzol)

cefinetaxole sodium (Zeegazone)

cefonocid sodium (Monocid)

cefoperazone sodium (Cefobid)

ceforanide (Precef)

cefotaxime sodium (Claforan)

cefotetan sodium (Cefotan, Apatef)

cefoxitin sodium (Mefoxin)

ceftazidime (Ceptaz, Fortaz, Tazicef, Tazidime)

ceftizoxime sodium (Kefurox, Zinacef)

ceftriaxone (Rocephin)

ceeftriaxone sodium (Rocephin)

cefuroxime-injectable (Kefurox, Zinacef)

cephradine-injectable (Velosef-injectable)

cephalothin sodium (Keflin, Seffin)

cephaapirin sodium (Cefadyl)

moxalactam (Moxam)

amoxicillin (Amoxil, Polymox)

amoxicillin and clavulanate (Augmentin)

ampicillin (Omnipen, Principen)

ampicillin and sulbactam (Unasyn)

azlocillin (Azlin)

bacammpicillin (Spectrobid)

carbenicillin (Geocillin, Geopen)

cefaclor (Ceclor)

cefdadroxil (Duricef, Ultracef)

cefamandole (Mandol)

cefazolin sodium (Ancef, Kefzol)

cefixine (Suprax)

cefmetazole sodium (Zefazone)

cefonicid (Monocid)

cefoperazone (Cefobid)

ceforanide (Precef)

cefotaxime (Claaforan)

cefotetan (Cefotan)

cefoxitin (Mefoxin)

cefprozil-injectable

cefprozil-oral (Cefzil)

ceftazidime (Ceptaz, Fortax)

ceftizoxime (Cefizox)

ceftriaxone (Rocephin)

cefuroxime (Ceftin, Zinacef)

cephalexin (Keflex, Keftab)

cephalothin (Keflin)

cephapirin (Cefadyl)

cephradine (Anspor, Velosef)

cloxacillin (Cloxapen, Tegopen)

cyclacillin

dicloxacillin (Dycill, Pathocil)

loracarbef-injectable

loracarbef-oral

methicillin (Staphcillin)

mezlocillin (Mezlin)

moxalactam (Moxam)

nafcillin (Nafcil, Unipen)

oxacillin (Bactocill, Prostaphlin)

penicillamine (Cuprimine, Depen)

penicillin G (Wycillin, Pentids, Bicillin LA)

penicillin V (Veetids, V-Cillin K)

piperacillin (Pipracil)

ticarcillin (Ticar)

ticarcillin and clavulanate (Timentin)

Other commercially available β-lactam antibiotics include imipenem (primaxin) which is typically given in combination with cilastatin (which prevents any degradation by renal enzymes). Other β-lactam antibiotics include meropenem (Meronem/Zeneca) and aztreonam (Azacetam). Other penems include biapenem, panipenem, carumonam, and ritipenam.

In accordance with the present invention the antibacterial efficacy of such β-lactam antibiotics can be enhanced, particularly in treatment of infections of β-lactamase-producing bacterial by co-administration (either separately or together in a combination pharmaceutical composition) of a non-β-lactam compound capable of inhibiting N-acetylated, α-linked acetic peptidase (NAALADase), the compound being administered in an amount effective to inhibit β-lactamase. In accordance with a related embodiment of the invention, there is provided an antibiotic composition comprising a β-lactam antibiotic, a non-β-lactam inhibitor of NAALADase in an amount effective to inhibit β-lactamase, and a pharmaceutically acceptable carrier.

Examples of known NAALADase inhibitors which can be utilized in accordance with the present invention include general metallo-peptidase inhibitors such as O-phenanthroline, metal chelators, such as ethylenediaminetetracetic acid (EDTA) and ethyleneglycol-bis(betaminoethylether)-N,N-tetracetic acid (EGTA) and peptide analogs such as quisqualic acid, aspartate glutamate (Asp-Glu), Glu-Glu, Gly-Glu, γ-Glu-Glu and beta-N-acetyl-L-aspartate-L-glutamate (β-NAAG). Other NAALADase inhibitors are the more recently described compounds of the formula

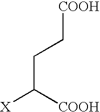

wherein X is RP(O)(OH)CH₂— [See U.S. Pat. No. 5,968,915 incorporated herein by reference]; RP(O)(OH)NH— [See U.S. Pat. No. 5,863,536 incorporated herein by reference]; RP(O)(OH)O— [See U.S. Pat. No. 5,795,877 incorporated herein by reference]; RN(OH)C(O)Y— or RC(O)NH(OH)Y wherein Y is CR₁R₂, NR₃ or O [See U.S. Pat. No. 5,962,521 incorporated herein by reference]; or X is RS(O)Y, RSO₂Y, or RS(O)(NH)Y wherein Y is CR₁R₂, NR₃ or O [See U.S. Pat. No. 5,902,817 incorporated herein by reference].

Exemplary of the NAALADase inhibitors described in U.S. Pat. No. 5,968,915 are as follows:

2-[[methylhydroxyphosphinyl]methyl]pentanedioic acid;

2-[[ethylhydroxyphosphinyl]methyl]pentanedioic acid;

2-[[propylhydroxyphosphinyl]methyl]pentanedioic acid;

2-[[butylhydroxyphosphinyl]methyl]pentanedioic acid;

2-[[cyclohexylhydroxyphosphinyl]methyl]pentanedioic acid;

2-[[phenylhydroxyphcsphinyl]methyl]pentanedioic acid;

2-[[2-(tetrahydrofuranyl)hydroxyphosphinyl]methyl]pentanedioic acid;

2-[[(2-tetrahydropyranyl)hydroxyphosphinyl]methyl]pentanedioic acid;

2-[[((4-pyridyl)methyl)hydroxyphosphinyl]methyl]pentanedioic acid;

2-[[((2-pyridyl)methyl)hydroxyphosphinyl]methyl]pentanedioic acid;

2-[[(phenylmethyl)hydroxyphosphinyl]methyl]pentanedioic acid;

2-[[((2-phenylethyl)methyl)hydroxyphosphinyl]methyl]pentanedioic acid;

2-[[((3-phenylpropyl)methyl)hydroxyphosphinyl]methyl]pentanedioic acid;

2-[[((3-phenylbutyl)methyl)hydroxyphosphinyl]methyl]pentanedioic acid;

2-[[((2-phenylbutyl)methyl)hydroxyphosphinyl]methyl]bpentanedioic acid;

2-[[(4-phenylbutyl)hydroxyphosphinyl]methyl]pentanedioic acid; and

2-[[(aminomethyl)hydroxyphosphinyl]methyl]pentanedioic acid.

Exemplary of the NAALADase inhibitors described in U.S. Pat. No. 5,863,536 are as follows:

N-[methylhydroxyphosphinyl]glutamic acid;

N-[ethylhydroxyphosphinyl]glutamic acid;

N-[propylhydroxyphosphinyl]glutamic acid;

N-[butylhydroxyphosphinyl]glutamic acid;

N-[phenylhydroxyphosphinyl]glutamic acid;

N-[(phenylmethyl)hydroxyphosphinyl]glutamic acid;

N-[((2-phenylethyl)methyl)hydroxyphosphinyl]glutamic acid; and

N-methyl-N-[phenylhydroxyphosphinyl]glutamic acid.

Exemplary of the NAALADase inhibitors described in U.S. Pat. No. 5,795,877 are as follows:

2-[[methylhydroxyphosphinyl]oxy]pentanedioic acid;

2-[[ethylhydroxyphosphinyl]oxy]pentanedioic acid;

2-[[propylhydroxyphosphinyl]oxy]pentanedioic acid;

2-[[butylhydroxyphosphinyl]oxy]pentanedioic acid;

2-[[phenylhydroxyphosphinyl]oxy]pentanedioic acid;

2-[[((4-pyridyl)methyl)hydroxyphosphinyl]oxy]pentanedioic acid;

2-[[((2-pyridyl)methyl)hydroxyphosphinyl]oxy]pentanedioic acid;

2-[[(phenylmethyl)hydroxyphosphinyl]oxy]pentanedioic acid; and

2[[((2-phenylethyl)methyl)hydroxyphosphinyl]oxy]pentanedioic acid.

Exemplary of the NAALADase inhibitors described in U.S. Pat. No. 5,962,521 are as follows:

2-[[(N-hydroxy)carbamoyl]methyl]pentanedioic acid;

2-[[(N-hydroxy-N-methyl)carbamoyl]methyl]pentanedioic acid;

2-[[(N-butyl-N-hydroxy)carbamoyl]methyl]pentanedioic acid;

2-[[(N-benzyl-N-hydroxy)carbamoyl]methyl]pentanedioic acid;

2-[[(N-hydroxy-N-phenyl)carbamoyl]methyl]pentanedioic acid;

2-[[(N-hydroxy-N-2-phenylethyl)carbamoyl]methyl]pentanedioic acid;

2-[[(N-ethyl-N-hydroxy)carbamoyl]methyl]pentanedioic acid;

2-[[(N-hydroxy-N-propyl)carbamoyl]methyl]pentanedioic acid;

2-[[(N-hydroxy-N-3-phenylpropyl)carbamoyl]methyl]pentanedioic acid;

2-[[(N-hydroxy-N-4-pyridyl)carbamoyl]methyl]pentanedioic acid;

2-[[(N-hydroxy)carboxamido]methyl]pentanedioic acid;

2-[[N-hydroxy(methyl)carboxamido]methyl]pentanedioic acid;

2-[[N-hydroxy(benzyl)carboxamido]methyl]pentanedioic acid;

2-[[N-hydroxy(phenyl)carboxamido]methyl]pentanedioic acid;

2-[[N-hydroxy(2-phenylethyl)carboxamido]methyl]pentanedioic acid;

2-[[N-hydroxy(ethyl)carboxamido]methyl]pentanedioic acid;

2-[[N-hydroxy(propyl)carboxamido]methyl]pentanedioic acid;

2-[[N-hydroxy(3-phenylpropyl)carboxamido]methyl]pentanedioic acid; and

2-[[N-hydroxy(4-pyridyl)carboxamido]methyl]pentanedioic acid.

Exemplary of the NAALADase inhibitors described in U.S. Pat. No. 5,902,817 are as follows:

2-[(sulfinyl)methyl]pentanedioic acid;

2-[(methylsulfinyl)methyl]pentanedioic acid;

2-[(ethylsulfinyl)methyl]pentanedioic acid;

2-[(propylsulfinyl)methyl]pentanedioic acid;

2-[(butylsulfonyl)methyl]pentanedioic acid;

2-[(phenylsulfinyl]methyl]pentanedioic acid;

2-[[(2-phenylethyl)sulfinyl]methyl]pentanedioic acid;

2-[[(3-phenylpropyl)sulfinyl]methyl]pentanedioic acid;

2-[[(4-pyridyl)sulfinyl]methyl]pentanedioic acid;

2-[(benzylsulfinyl)methyl]pentanedioic acid;

2-[(sulfonyl)methyl]pentanedioic acid;

2-[(methylsulfonyl)methyl]pentanedioic acid;

2-[(ethylsulfonyl)methyl]pentanedioic acid;

2-[(propylsulfonyl)methyl]pentanedioic acid;

2-[(butylsulfonyl)methyl]pentanedioic acid;

2-[(phenylsulfonyl]methyl]pentanedioic acid;

2-[[(2-phenylethyl)sulfonyl]methyl]pentanedioic acid;

2-[[(3-phenylpropyl)sulfonyl]methyl]pentanedioic acid;

2-[[(4-pyridyl)sulfonyl]methyl]pentanedioic acid;

2-[(benzylsulfonyl)methyl]pentanedioic acid;

2-[(sulfoximinyl)methyl]pentanedioic acid;

2-[(methylsulfoximinyl)methyl]pentanedioic acid;

2-[(ethylsulfoximinyl)methyl]pentanedioic acid;

2-[(propylsulfoximiyl)methyl]pentanedioic acid;

2-[(butylsulfoximinyl)methyl]pentanedioic acid;

2-[(phenylsulfoximinyl]methyl]pentanedioic acid;

2-[[(2-phenylethyl)sulfoximinyl]methyl]pentanedioic acid;

2-[[(3-phenylpropyl)sulfoximinyl]methyl]pentanedioic acid;

2-[[(4-pyridyl)sulfoximinyl]methyl]pentanedioic acid; and

2-[(benzylsulfoximinyl)methyl]pentanedioic acid.

Other compounds have been reported in the literature to have NAALADase inhibitory activity. The foregoing list of NAALADase inhibitors is intended to be exemplary of NAALADase inhibitors that can be utilized in the method and composition embodiments of the present invention. The amount of the non-β-lactam NAALADase inhibitor appropriate for use in combination with β-lactam antibiotics depends not only on the level of β-lactamase inhibitory activity exhibited by the NAALADase inhibitor, but also on the intended route of administration, and the pharmacokinetics of the inhibitor itself. Typically the weight ratio of β-lactam antibiotic to NAALADase inhibitor will be about 1:1 to about 50:1, more typically about 2:1 to about 30:1. Typical unit dosages of β-lactam antibiotics range from about 200 mg to about 2 g/dose. The β-lactam antibiotic and the NAALADase inhibitor can be administered independently, optionally by different routes of administration, or in combination in an antibiotic composition comprising a β-lactam antibiotic, the NAALADase inhibitor, and a pharmaceutically acceptable carrier. The compositions can be formulated, for example, for oral administration in such forms as tablets, capsules, caplets, dispersible powders, granules, lozenges, mucosal patches, sachets, and the like. Suitable carriers for such dosage forms include, for example, starch, lactose or trehalose, alone or in combination with one or more formulation excipients. Optionally, oral dosage forms such as tablets, caplets or capsules can be enterically coated to minimize hydrolysis/degradation of the active components in the stomach. In another embodiment, the dosage form is formulated for oral administration in a prolonged release dosage form, designed to release the active ingredients over a predetermined period of time. The methods and carrier components useful for preparing such prolonged release compositions are well known in the art.

The antibiotic compositions in accordance with this invention alternatively can be formulated for parenteral administration, including subcutaneous administration, intraperitoneal administration, intramuscular administration, intrathecal administration and intravenous administration. Such parenteral dosage forms are typically in the form of aqueous solutions or dispersions utilized in a pharmaceutically acceptable carrier such as isotonic saline, 5% glucose or other well known pharmaceutically accepted liquid carrier compositions. The antibiotic compositions suitable for injectable use include sterile aqueous solutions or dispersions and sterile powders or lyophilizates for the extemporaneous preparation of sterile injectable solutions or dispersions.

Parenteral dosage forms of the present antibiotic compositions can also be formulated as injectable prolonged release formulations in which the β-lactam antibiotic and the NAALADase inhibitor are combined with one or more natural or synthetic biodegradable or biodispersible polymers such as carbohydrates, including starches, gums and etherified or esterified cellulosic derivatives, polyethers, polyesters, polyvinyl alcohols, gelatins or alginates. Such dosage formulations can be prepared, for example, in the form of microsphere suspensions, gels (of hydrophilic or hydrophobic constitution), or shaped-polymer matrix implants that are well known in the art for their function as “depot-type” drug delivery systems that provide prolonged release of the biologically reactive components. Such compositions are prepared using art recognized formulation techniques, and can be designed for any of a wide variety of drug release profiles.

The administration of β-lactam antibiotics and NAALADase inhibitors in accordance with this invention, either independently or in an antibiotic composition of the invention, can be intermittent or at a gradual, continuous, constant or controlled rate to a patient in need of treatment. In addition, the daily dosage amount can be divided and administered in two or more daily doses depending on patient condition and environment. The optimal dosage amounts and dosage form for implementing the present method in accordance with this invention is dependent not only on the absorption and pharmacokinetic properties of the β-lactam antibiotic and the NAALADase inhibitor, but also on patient and patient condition and adjustable within reasonable ranges in the judgment of the attending physician. The formulation is typically administered over a period of time sufficient to eliminate the bacterial source of infection in the patient undergoing treatment and administration may be continued using the same or an attenuated dosage protocol for prophylaxis.

To treat an animal suffering from a bacterial infection, including β-lactam-antibiotic-resistant bacterial infections, an effective amount of a compound capable of inhibiting NAALADase or a pharmaceutically-acceptable salt thereof, is administered to the animal, in combination with a β-lactam antibiotic. Effective dosage forms, modes of administration and dosage amounts of the NAALADase inhibitor, may be determined empirically, and making such determinations is within the skill in the art. It is understood by those skilled in the art that the dosage amount will vary with the activity of the particular compound employed, the severity of the bacterial infection, whether the bacterial infection is resistant to treatment with β-lactam antibiotics, the route of administration, the rate of excretion of the compound, the duration of the treatment, the identity of any other drugs being administered to the animal, the age, size and species of the animal, and like factors well known in the medical and veterinary arts. In general, a suitable daily dose will be that amount which is the lowest dose effective to produce a therapeutic effect. The total daily dosage will be determined by an attending physician or veterinarian within the scope of sound medical judgment. If desired, the effective daily dose of a composition of the present invention, may be administered as two, three, four, five, six or more sub-doses, administered separately at appropriate intervals throughout the day. Treatment of a bacterial infection, including β-lactam-antibiotic-resistant bacterial infections, according to the invention, includes mitigation, as well as elimination, of the infection.

Animals that can be treated in accordance with the invention include mammals. Mammals that can be treated according to the invention include dogs, cats, other domestic animals, and humans.

The present composition comprising a non-β-lactam NAALADase inhibitor and a β-lactam antibiotic may be administered to a patient by any suitable route of administration, including orally, nasally, rectally, intravaginally, parenterally, intracisternally and topically, as by powders, ointments or drops, including buccally and sublingually. The preferred routes of administration are oral and parenteral administration.

While it is possible for the active ingredient(s) to be administered alone, it is preferable to administer the active ingredient(s) as a pharmaceutical formulation (composition). The pharmaceutical compositions of the invention comprise the active ingredient(s) in admixture with one or more pharmaceutically-acceptable carriers and, optionally, with one or more other compounds, drugs or other materials. Each carrier must be “acceptable” in the sense of being compatible with the other ingredients of the formulation and not injurious to the patient.

Pharmaceutical formulations of the present invention include those suitable for oral, nasal, topical (including buccal and sublingual), rectal, vaginal and/or parenteral administration. Regardless of-the route of administration selected, the active ingredient(s) are formulated into pharmaceutically-acceptable dosage forms by conventional methods known to those skilled in the art.

The amount of the active ingredient(s) which will be combined with a carrier material to produce a single dosage form will vary depending upon the host being treated, the particular mode of administration and all of the other factors described above. The amount of the active ingredient(s) which will be combined with a carrier material to produce a single dosage form will generally be that amount of the active ingredient(s) which is the lowest dose effective to produce a therapeutic effect.

Methods of preparing pharmaceutical formulations or compositions include the step of bringing the active ingredient(s) into association with the carrier and, optionally, one or more accessory ingredients. In general, the formulations are prepared by uniformly and intimately bringing the active ingredient(s) into association with liquid carriers, or finely divided solid carriers, or both, and then, if necessary, shaping the product.

Formulations of the invention suitable for oral administration may be in the form of capsules, cachets, pills, tablets, lozenges (using a flavored basis, typically sucrose and acacia or tragacanth), powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia) and/or as mouth washes and the like, each containing a predetermined amount of the active ingredient(s). The active ingredient(s) may also be administered as a bolus, electuary or paste.

In solid dosage forms of the invention for oral administration (capsules, tablets, pills, dragees, powders, granules and the like), the active ingredient(s) is/are mixed with one or more pharmaceutically-acceptable carriers, such as sodium citrate or dicalcium phosphate, and/or any of the following: (1) fillers or extenders, such as starches, lactose, sucrose, glucose, mannitol, and/or silicic acid; (2) binders, such as, for example, carboxymethylcellulose, alginates, gelatin, polyvinyl pyrrolidone, sucrose and/or acacia; (3) humectants, such as glycerol; (4) disintegrating agents, such as agar-agar, calcium carbonate, potato or tapioca starch, alginic acid, certain silicates, and sodium carbonate; (5) solution retarding agents, such as paraffin; (6) absorption accelerators, such as quaternary ammonium compounds; (7) wetting agents, such as, for example, cetyl alcohol and glycerol monostearate; (8) absorbents, such as kaolin and bentonite clay; (9) lubricants, such as talc, calcium stearate, magnesium stearate, solid polyethylene glycols, sodium lauryl sulfate, and mixtures thereof; and (10) coloring agents. In the case of capsules, tablets and pills, the pharmaceutical compositions may also comprise buffering agents. Solid compositions of a similar type may also be employed as fillers in soft and hard-filled gelatin capsules using such excipients as lactose or milk sugars, as well as high molecular weight polyethylene glycols and the like.

A tablet may be made by compression or molding, optionally with one or more accessory ingredients. Compressed tablets may be prepared using binder (for example, gelatin or hydroxypropylmethyl cellulose), lubricant, inert diluent, preservative, disintegrant (for example, sodium starch glycolate or cross-linked sodium carboxymethyl cellulose), surface-active or dispersing agent. Molded tablets may be made by molding in a suitable machine a mixture of the powdered active ingredient(s) moistened with an inert liquid diluent.

The tablets, and other solid dosage forms of the pharmaceutical compositions of the present invention, such as dragees, capsules, pills and granules, may optionally be scored or prepared with coatings and shells, such as enteric coatings and other coatings well known in the pharmaceutical-formulating art. They may also be formulated so as to provide slow or controlled release of the active ingredient(s) therein using, for example, hydroxypropylmethyl cellulose in varying proportions to provide the desired release profile, other polymer matrices, liposomes and/or microspheres. They may be sterilized by, for example, filtration through a bacteria-retaining filter. These compositions may also optionally contain opacifying agents and may be of a composition capable of releasing the active ingredients only, or preferentially, in a certain portion of the gastrointestinal tract, optionally, in a delayed manner. Examples of embedding compositions which can be used include polymeric substances and waxes. The active ingredient(s) can also be in microencapsulated form.

Liquid dosage forms for oral administration of the active ingredients include pharmaceutically-acceptable emulsions, microemulsions, solutions, suspensions, syrups and elixirs. In addition to the active ingredient(s), the liquid dosage forms may contain inert diluents commonly used in the art, such as, for example, water or other solvents, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan, and mixtures thereof.

Besides inert diluents, the oral compositions can also include adjuvants such as wetting agents, emulsifying and suspending agents, sweetening, flavoring, coloring, perfuming and preservative agents.

Suspensions, in addition to the active ingredient(s), may contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

Formulations of the pharmaceutical compositions of the invention for rectal or vaginal administration may be presented as a suppository, which may be prepared by mixing the active ingredient(s) with one or more suitable nonirritating excipients or carriers comprising, for example, cocoa butter, polyethylene glycol, a suppository wax or salicylate and which is solid at room temperature, but liquid at body temperature and, therefore, will melt in the rectum or vaginal cavity and release the active ingredient(s). Formulations of the present invention which are suitable for vaginal administration also include pessaries, tampons, creams, gels, pastes, foams or spray formulations containing such carriers as are known in the art to be appropriate.

Dosage forms for the topical or transdermal administration of the active ingredient(s) include powders, sprays, ointments, pastes, creams, lotions, gels, solutions, patches and inhalants. The active ingredient(s) may be mixed under sterile conditions with a pharmaceutically-acceptable carrier, and with any buffers, or propellants which may be required.

The ointments, pastes, creams and gels may contain, in addition to the active ingredient(s), excipients, such as animal and vegetable fats, oils, waxes, paraffins, starch, tragacanth, cellulose derivatives, polyethylene glycols, silicones, bentonites, silicic acid, talc and zinc oxide, or mixtures thereof.

Powders and sprays can contain, in addition to the active ingredient(s), excipients such as lactose, talc, silicic acid, aluminum hydroxide, calcium silicates and polyamide powder, or mixtures of these substances. Sprays can additionally contain customary propellants such as chlorofluorohydrocarbons and volatile unsubstituted hydrocarbons, such as butane and propane.

Transdermal patches have the added advantage of providing controlled delivery of the active ingredient(s) to the body. Such dosage forms can be made by dissolving, dispersing or otherwise incorporating the active ingredient(s) in a proper medium, such as an elastomeric matrix material. Absorption enhancers can also be used to increase the flux of the active ingredient(s) across the skin. The rate of such flux can be controlled by either providing a rate-controlling membrane or dispersing the active ingredient(s) in a polymer matrix or gel.

Pharmaceutical compositions of this invention suitable for parenteral administration comprise the active ingredient(s) in combination with one or more pharmaceutically-acceptable sterile isotonic aqueous or non-aqueous solutions, dispersions, suspensions or emulsions, or sterile powders which may be reconstituted into sterile injectable solutions or dispersions just prior to use, which may contain antioxidants, buffers, solutes which render the formulation isotonic with the blood of the intended recipient or suspending or thickening agents.

Examples of suitable aqueous and non-aqueous carriers which may be employed in the pharmaceutical compositions of the invention include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

These compositions may also contain adjuvants such as wetting agents, emulsifying agents and dispersing agents. It may also be desirable to include isotonic agents, such as sugars, sodium chloride, and the like in the compositions. In addition, prolonged absorption of the injectable pharmaceutical form may be brought about by the inclusion of agents which delay absorption such as aluminum monostearate and gelatin.

In some cases, in order to prolong the effect of the active ingredient(s), it is desirable to slow the absorption of the drug from subcutaneous or intramuscular injection. This may be accomplished by the use of a liquid suspension of crystalline or amorphous material having poor water solubility. The rate of absorption of the active ingredient(s) then depends upon its/their rate of dissolution which, in turn, may depend upon crystal size and crystalline form. Alternatively, delayed absorption of parenterally-administered active ingredient(s) is accomplished by dissolving or suspending the active ingredients in an oil vehicle.

Injectable depot forms are made by forming microencapsule matrices of the active ingredient(s) in biodegradable polymers such as polylactide-polyglycolide. Depending on the ratio of the active ingredient(s) to polymer, and the nature of the particular polymer employed, the rate of release of the active ingredient(s) can be controlled. Examples of other biodegradable polymers include poly(orthoesters) and poly(anhydrides). Depot injectable formulations are also prepared by entrapping the active ingredient(s) in liposomes or microemulsions which are compatible with body tissue. The injectable materials can be sterilized for example, by filtration through a bacterial-retaining filter.

The formulations may be presented in unit-dose or multi-dose sealed containers, for example, ampules and vials, and may be stored in a lyophilized condition requiring only the addition of the sterile liquid carrier, for example water for injection, immediately prior to use. Extemporaneous injection solutions and suspensions may be prepared from sterile powders, granules and tablets of the type described above.

The pharmaceutical compositions of the present invention may also be used in the form of veterinary formulations, including those adapted for the following: (1) oral administration, for example, drenches, aqueous or non-aqueous solutions or suspensions, tablets, boluses, powders, granules or pellets for admixture with feed stuffs, pastes for application to the tongue; (2) parenteral administration, for example, by subcutaneous, intramuscular or intravenous injection as, for example, a sterile solution or suspension or, when appropriate, by intramammary injection where a suspension or solution is introduced into the udder of the animal via its teat; (3) topical application, for example, as a cream, ointment or spray applied to the skin; or (4) intravaginally, for example, as a pessary, cream or foam.

The following is illustrative of antibiotic compositions in accordance with the present invention. The compositions are prepared using the specified β-lactam antibiotic and NAALADase inhibitor components at three unique mass ratios of β-lactam antibiotic to NAALADase inhibitor (10:1, 2:1; and 1:1) using isotonic sterile saline as the carrier (for parenteral administration).

ANTIBIOTIC COMPOSITIONS
NAALADase Inhibitor

| | | | (C) |
|---|---|---|---|
| | (A) | (B) | 2-(phosphonomethyl) |
| Antibiotic | β-NAAG | Quisqualic acid | pentanedioic acid |
| I. Amoxicillin | ✓ | ✓ | ✓ |
| II. Ticaricillin | ✓ | ✓ | ✓ |
| III. Cefoperazone | ✓ | ✓ | ✓ |
| IV. Piperacillin | ✓ | ✓ | ✓ |
| V. Ampicillin | ✓ | ✓ | ✓ |

1. An antibiotic composition comprising a β-lactam antibiotic and an inhibitor of N-acetylated α-linked acidic dipeptidase in an amount effective to inhibit β-lactamase and a pharmaceutically acceptable carrier therefor, provided that the chemical structure of said inhibitor does not include a β-lactam ring.

2. The composition of claim 1 in a dosage form for oral administration.

3. The composition of claim 1 in a parenteral dosage form.

4. A method for treating bacterial infection in a warm-blooded vertebrate comprising the steps of administering to said vertebrate an antibiotically effective amount of a β-lactam antibiotic and administering an inhibitor of N-acetylated α-linked acidic dipeptidase in an amount effective to inhibit β-lactamase, provided that the chemical structure of the inhibitor does not include a β-lactam ring.

5. The method of claim 4 wherein the β-lactam antibiotic and the inhibitor are administered separately.

6. The method of claim 4 wherein the β-lactam antibiotic and the inhibitor are administered in combination as an antibiotic composition.

7. The composition of claim 1 wherein the β-lactam antibiotic is selected from the group consisting of penicillin and cephalosporins.

8. The method of claim 4 wherein the β-lactam antibiotic is selected from the group consisting of penicillins and cephalosporins.
